(19) United States
(12) Patent Application Publication
(10) Pub. No.: US 2012/0010312 A1
(43) Pub. Date: Jan. 12, 2012

(54) POLYMERIC FLAME RETARDANT

(75) Inventors: Marco Balbo Block, Osnabrueck (DE); Jens Ferbitz, Osnabrueck (DE); Christoph Fleckenstein, Freigericht (DE); Anna Cristadoro, Heppenheim (DE); Bernd Bruchmann, Freinsheim (DE); Klemens Massonne, Bad Duerkheim (DE)

(73) Assignee: BASF SE, Ludwigshafen (DE)

(21) Appl. No.: 13/111,289

(22) Filed: May 19, 2011

Related U.S. Application Data
(60) Provisional application No. 61/346,918, filed on May 21, 2010.

(51) Int. Cl. C08L 75/04; C08K 5/53; C07C 69/96; C08J 9/00

(52) U.S. Cl. 521/130; 524/139; 558/268

(57) ABSTRACT

The present invention relates to a polycarbonate comprising at least one phosphorus-containing group, to the use of the polycarbonate as flame retardant, to a process for producing a polyurethane by using this polycarbonate, and to a polyurethane obtain-able by this process.

The present invention relates to a polycarbonate comprising at least one phosphorus-containing group, to the use of the polycarbonate as flame retardant, to a process for producing a polyurethane by using this polycarbonate comprising at least one phosphorus-containing group, and to a polyurethane obtainable by this process.

There are many different methods for providing flame retardancy to polymers, in particular polyurethanes, and very particularly polyurethane foams. A first method is formation of a crust to prevent the flame from reaching the combustible material. Thermal hydrolysis products remove oxygen from the polymer matrix and lead to formation of a layer of carbon on the surface of the polymer. This layer of carbon prevents the flame from causing either thermal or oxidative decomposition of the plastic located below the layer. The term used is intumescence. Phosphorus-containing compounds, and among these organophosphorus compounds, are widely used to form a carbonized crust in the event of a fire. Organophosphorus flame retardants are mostly based on phosphate esters, on phosphonate esters, or on phosphite esters.

Halogenated compounds are also used as flame retardants. In contrast to phosphorus-containing flame retardants, these act within the gas phase of the flame. Low-reactivity free halogen radicals here scavenge various high-reactivity free radicals derived from degradation products of the polymer, thus inhibiting fire propagation by way of free radicals. Bromine-containing flame retardants are particularly effective here. Another particularly effective flame retardant is trichloroisopropyl phosphate (TCPP), which comprises not only phosphate but also the halogen chlorine, and thus acts by way of both of the mechanisms described above.

However, halogenated flame retardants, in particular bromine-containing flame retardants, are undesirable for toxicological, environmental, and regulatory reasons. Halogen-containing flame retardants also increase smoke density in the event of a fire. Attempts are therefore being made to achieve general avoidance of halogen-containing flame retardants.

Examples of known halogen-free flame retardants are solid flame retardants such as melamine or ammonium polyphosphate. These solid particles have adverse effects on the polymers, in particular on the properties of polyurethane foams. Solid flame retardants also specifically cause problems during the production of the polyurethanes. By way of example, the production of polyurethanes preferably uses liquid starting materials, including those in the form of solutions. The use of solid particles leads to separation phenomena in the mixtures usually used for polyurethane production, and the life of batches is therefore only about one day. The solid flame retardant particles moreover abrade the metering units, for example in the foam plants. Said flame retardants also have an adverse effect on the chemical processes during the foaming process and have an adverse effect on the properties of the foam.

Many liquid flame retardants, such as triethyl phosphate (TEP) or diethyl ethane-phosphonate (DEEP), contribute by way of example to emissions from the plastics, giving these an unpleasant odor. The liquid flame retardants moreover have an adverse effect on the foaming reaction during the production of polyurethane foams, and also on the properties of the foams, for example mechanical properties. Known liquid flame retardants also frequently act as plasticizers.

In order to counter problems with emissions, incorporatable flame retardants have been developed for polyurethanes. Incorporatable flame retardants, such as Exolit® OP560 from Clariant, generally have functionality ≦2 with respect to isocyanates and frequently reduce crosslinking density in polyurethane foams, thus impairing the properties of the foam, in particular in rigid polyurethane foam.

WO 2003104374 A1, WO 2004076509 A2, and WO 2005052031 A1 describe the use of phosphonic-acid-reacted, hyperbranched polyacrylonitrile polyacrylamide, polyamide, and polyamine as rust preventer, lubricant, textile additive, and flame retardant. Said compounds are not suitable for use for polyurethanes and in particular polyurethane foams, since the nitrogen-containing structures severely affect the catalysis of the foam-formation process.

In EP 474076 B1, Bayer AG describes highly branched polyphosphates as flame retardants for polycarbonates. The structure of these materials, made of aromatic dihydroxy compounds and of phosphonate esters or polyphosphorus compounds, gives them poor solubility in the polyols used for polyurethane production, and this makes it difficult to process this class of compound in polyurethanes.

WO 2007066383 describes hyperbranched polyesters which were reacted with phosphorus compounds, such as 9,10-dihydro-9-oxa-10-phosphaphenanthrene 10-oxide, and also the use of these as flame retardants for resins. The low thermal and hydrolytic stability of the ester groups is disadvantageous.

It was therefore an object of the present invention to provide a halogen-free flame retardant which can also be used in the production of polyurethane.

Another object of the present invention was to provide flame retardants whose use does not lead to emissions in polymers, in particular in polyurethanes, and specifically in polyurethane foams, and whose use in polymers, in particular in polyurethanes and specifically in polyurethane foams, does not lead to impairment of properties, in particular of mechanical properties.

Another object of the present invention was to provide a flame retardant which can be used not only during the extrusion of thermoplastics but also during the production of crosslinking plastics.

These objects of the invention are achieved via a polycarbonate comprising at least one phosphorus-containing group, the use of the polycarbonate as flame retardant, a process for producing a polyurethane by using this polycarbonate comprising at least one phosphorus-containing group, and a polyurethane obtainable by this process.

Polycarbonates here are compounds obtainable from the reaction of alcohols or phenols with phosgene, or from the transesterification of alcohols or phenols with dialkyl or diaryl carbonates. Polycarbonates are therefore formally esters of carbonic acid. For the purposes of the invention, the term polycarbonates is used when the molecule has at least 3, preferably at least 5, and in particular at least 10, —O—(CO)—O— groups. Copolymers are also hereinafter termed polycarbonates when they have the abovementioned minimum number of —O—(CO)—O— groups. In one embodiment of the present invention, at least 50%, particularly preferably at least 70%, and in particular at least 90%, of the end groups in polycarbonates of the invention are OH groups.

The production of polycarbonates is well known and widely described, for example in Becker/Braun, Kunststoff-Handbuch [Plastics handbook] volume 3/1, Polycarbonate, Polyacetale, Polyester, Celluloseester [Polycarbonates, polyacetals, polyesters, cellulose esters], Carl-Hanser-Verlag, Munich 1992, pages 118 to 119, and “Ullmann's Encyclopedia of Industrial Chemistry”, 6th edition, 2000 Electronic Release, Verlag Wiley-VCH.

For the purposes of the present invention, alongside the linear polycarbonates, use is preferably made of branched or hyperbranched polycarbonates. Branched or hyperbranched polycarbonates are also known and described by way of example in WO 9850453 and WO 2005026234.

For the purposes of the invention, it is particularly preferable to use hyperbranched polycarbonates which can be produced either by reacting at least one organic carbonate (A) of the general formula RO[(CO)O]ₙR with at least one aliphatic, aliphatic/aromatic, or aromatic alcohol (B) which has at least 3 OH groups, with elimination of alcohols ROH, where each R, independently of the others, is a straight-chain or branched aliphatic, aromatic/aliphatic, or aromatic hydrocarbon radical having from 1 to 20 carbon atoms, and where the radicals R can also have bonding to one another to form a ring, and n is an integer from 1 to 5, or by reacting phosgene, diphosgene, or triphosgene with said aliphatic or aromatic alcohol (B) with elimination of hydrogen chloride. The conduct of the reaction here is preferably such that the ratio of the compounds comprising OH groups to phosgene or carbonate gives an excess of OH groups present. The use of organic carbonate (A) is preferred here to the use of phosgene, diphosgene, or triphosgene. The average OH functionality of all of the alcohols with which the organic carbonate (A) is reacted here is preferably greater than 2.

For the purposes of this invention, hyperbranched polycarbonates are uncrosslinked macromolecules which have —O—(CO)—O— groups and which have both structural and molecular nonuniformity. On the one hand, they can have a structure that derives from a central molecule by analogy with dendrimers, but having nonuniform chain length of the branches. On the other hand, they can also have linear structure, having functional pendent groups, or else, in the form of a combination of the two extremes, can have linear and branched portions of the molecule. For the definition of dendrimers and of hyperbranched polymers, see also P. J. Flory, J. Am. Chem. Soc. 1952, 74, 2718 and H. Frey et al., Chem. Eur. J. 2000, 6, No. 14, 2499.

In the context of the present invention, “hyperbranched” means that the degree of branching (DB) is from 10 to 99.9%, preferably from 20 to 99%, particularly preferably from 20 to 95%. “Dendrimeric” in the context of the present invention means that the degree of branching is from 99.9 to 100%. For the definition of “degree of branching”, see H. Frey et al., Acta Polym. 1997, 48, 30.

Each of the radicals R of the organic carbonates (A) used as starting material of the general formula RO[(CO)O]ₙR is, independently of the others, a straight-chain or branched, aliphatic, aromatic/aliphatic, or aromatic or heteroaromatic hydrocarbon radical having from 1 to 20 carbon atoms. The two radicals R can also have bonding to one another to form a ring. The radical is preferably an aliphatic hydrocarbon radical and particularly preferably a straight-chain or branched alkyl radical having from 1 to 5 carbon atoms, or a substituted or unsubstituted phenyl radical.

The carbonates A) can preferably be simple carbonates of the general formula RO(CO)OR, so that in this case n is 1.

n is generally an integer from 1 to 5, preferably from 1 to 3.

By way of example, dialkyl or diaryl carbonates may be prepared from the reaction of aliphatic, araliphatic, or aromatic alcohols, preferably monoalcohols, with phosgene. They may also be prepared by way of oxidative carbonylation of the alcohols or phenols by means of CO in the presence of noble metals, oxygen, or NOₓ. In relation to preparation methods for diaryl or dialkyl carbonates, see also “Ullmann's Encyclopedia of Industrial Chemistry”, 6th edition, 2000 Electronic Release, Verlag Wiley-VCH.

Examples of suitable carbonates A) comprise aliphatic, aromatic/aliphatic or aromatic carbonates, such as ethylene carbonate, propylene 1,2- or 1,3-carbonate, diphenyl carbonate, ditolyl carbonate, dixylyl carbonate, dinaphthyl carbonate, ethyl phenyl carbonate, dibenzyl carbonate, dimethyl carbonate, diethyl carbonate, dipropyl carbonate, dibutyl carbonate, diisobutyl carbonate, dipentyl carbonate, dihexyl carbonate, dicyclohexyl carbonate, diheptyl carbonate, dioctyl carbonate, didecyl carbonate, or didodecyl carbonate.

Examples of carbonates A) where n is greater than 1 comprise dialkyl dicarbonates, such as di(tert-butyl)dicarbonate, or dialkyl tricarbonates, such as di(tert-butyl)tricarbonate.

It is preferable to use aliphatic carbonates, in particular those in which the radicals comprise from 1 to 5 carbon atoms, e.g. dimethyl carbonate, diethyl carbonate, dipropyl carbonate, dibutyl carbonate, or diisobutyl carbonate, or the aromatic carbonate diphenyl carbonate.

The organic carbonates are reacted with at least one aliphatic or aromatic alcohol (B) which has at least 3 OH groups, or with mixtures of two or more different alcohols. The average OH functionality of the mixture here is greater than 2, preferably greater than 2.5.

Examples of compounds having at least three OH groups comprise glycerol, trimethylolmethane, trimethylolethane, trimethylolpropane, 1,2,4-butanetriol, tris(hydroxymethyl)amine, tris(hydroxyethyl)amine, tris(hydroxypropyl)amine, pentaerythritol, diglycerol, triglycerol, polyglycerols, bis(trimethylolpropane), tris(hydroxymethyl)isocyanurate, tris(hydroxyethyl)isocyanurate, phloroglucinol, trihydroxytoluene, trihydroxydimethylbenzene, phloroglucides, hexahydroxybenzene, 1,3,5-benzenetrimethanol, 1,1,1-tris(4′-hydroxyphenyl)methane, 1,1,1-tris(4′-hydroxyphenyl)ethane, or sugars, e.g. glucose, sugar derivatives, trihydric or higher polyfunctional polyetherols based on trihydric or higher polyfunctional alcohols and ethylene oxide, propylene oxide, or butylene oxide, or a mixture thereof, or polyesterols. Particular preference is given here to glycerol, trimethylolethane, trimethylolpropane, 1,2,4-butanetriol, pentaerythritol, and also their polyetherols based on ethylene oxide or propylene oxide.

These polyfunctional alcohols (B) may also be used in a mixture with dihydric alcohols (B′), with the proviso that the average total OH functionality of all of the alcohols used is greater than 2. Examples of suitable compounds having two OH groups comprise ethylene glycol, diethylene glycol, triethylene glycol, 1,2- and 1,3-propanediol, dipropylene glycol, tripropylene glycol, neopentyl glycol, 1,2-, 1,3-, and 1,4-butanediol, 1,2-, 1,3-, and 1,5-pentanediol, hexanediol, cyclopentanediol, cyclohexanediol, cyclohexanedimethanol, bis(4-hydroxycyclohexyl)methane, bis(4-hydroxycyclohexyl)-ethane, 2,2-bis(4-hydroxycyclohexyl)propane, 1,1-bis(4-hydroxyphenyl)-3,3,5-tri-methylcyclohexane, resorcinol, hydroquinone, 4,4′-dihydroxybiphenyl, bis(4-hydroxy-phenyl)sulfide, bis(4-hydroxyphenyl)sulfone, bis(hydroxymethyl)benzene, bis-(hydroxymethyl)toluene, bis(p-hydroxyphenyl)methane, bis(p-hydroxyphenyl)ethane, 2,2-bis(p-hydroxyphenyl)propane, 1,1-bis(p-hydroxyphenyl)cyclohexane, dihydroxy-benzophenone, dihydric polyether polyols based on ethylene oxide, propylene oxide, butylene oxide, or mixtures of these, polytetrahydrofuran, polycaprolactone, or polyesterols based on diols and dicarboxylic acids.

The diols serve for fine adjustment of the properties of the polycarbonate. If use is made of dihydric alcohols, the ratio of dihydric alcohols (B′) to the at least trihydric alcohols (B) is set by the person skilled in the art and depends on the desired properties of the polycarbonate. The amount of the alcohol(s) (B′) is generally from 0 to 39.9 mol %, based on the total amount of all of the alcohols (B) and (B′) taken together. The amount is preferably from 0 to 35 mol %, particularly preferably from 0 to 25 mol %, and very particularly preferably from 0 to 10 mol %.

The reaction of phosgene, diphosgene, or triphosgene with the alcohol or alcohol mixture generally takes place with elimination of hydrogen chloride, and the reaction of the carbonates with the alcohol or alcohol mixture to give the inventive high-functionality highly branched polycarbonate takes place with elimination of the monofunctional alcohol or phenol from the carbonate molecule.

As polyfunctional alcohols (B) and as difunctional alcohols (B′) it is preferable to use more than 70 mol %, particularly more than 90 mol %, based on the total molar amount of the alcohols used, and in particular exclusively aliphatic alcohols. It is moreover preferable that the polycarbonates of the invention comprise no aromatic constituents in the carbonate skeleton.

The high-functionality highly branched polycarbonates formed by the inventive process have termination by hydroxy groups and/or by carbonate groups and, respectively, carbamoyl chloride groups after the reaction, i.e. with no further modification. They have good solubility in various solvents, e.g. in water, alcohols, such as methanol, ethanol, butanol, alcohol/water mixtures, acetone, 2-butanone, ethyl acetate, butyl acetate, methoxypropyl acetate, methoxyethyl acetate, tetrahydrofuran, dimethylformamide, dimethylacetamide, N-methylpyrrolidone, ethylene carbonate, or propylene carbonate.

For the purposes of this invention, a high-functionality polycarbonate is a product which, alongside the carbonate groups that form the skeleton of the polymer, has at least three, preferably at least six, more preferably at least ten, terminal or pendent functional groups. The functional groups are carbonate groups or carbamoyl chloride groups and/or OH groups, where the proportion of OH groups is preferably at least 50%, particularly preferably at least 70%, and in particular at least 90%, based in each case on the amount of terminal or pendent functional groups. In principle, there is no upper restriction on the number of the terminal or pendant functional groups, but products with a very large number of functional groups can have undesired properties, for example high viscosity or poor solubility. The high-functionality polycarbonates of the present invention mostly have no more than 500 terminal or pendent functional groups, preferably no more than 100 terminal or pendent functional groups.

In the production of the high-functionality polycarbonates, the ratio of the compounds comprising OH groups to phosgene or carbonate is preferably adjusted in such a way that the simplest resultant condensate comprises an average of either one carbonate or carbamoyl chloride group and more than one OH group or one OH group and more than one carbonate or carbamoyl chloride group.

The reaction to give the hyperbranched polycarbonate usually takes place at a temperature of from 0 to 300° C., preferably from 0 to 250° C., particularly preferably from 60 to 200° C., and very particularly preferably from 60 to 160° C., in bulk or in solution. It is possible in general here to use any of the solvents which are inert with respect to the respective starting materials. It is preferable to use organic solvents, such as decane, dodecane, benzene, toluene, chlorobenzene, xylene, dimethylformamide, dimethyl-acetamide, or solvent naphtha.

In one preferred embodiment, the condensation reaction is carried out in bulk. In order to accelerate the reaction, the monofunctional alcohol liberated during the reaction or the phenol ROH can be removed from the reaction equilibrium, for example by distillation, if appropriate at reduced pressure.

If removal by distillation is intended, it is generally advisable to use carbonates which during the reaction liberate alcohols or phenols ROH with boiling point below 140° C. at the prevailing pressure.

Catalysts or catalyst mixtures may also be added in order to accelerate the reaction. Suitable catalysts are compounds which catalyze esterification or transesterification reactions, examples being alkali metal hydroxides, alkali metal carbonates, alkali metal hydrogencarbonates, preferably of sodium, of potassium, or of cesium, tertiary amines, guanidines, ammonium compounds, phosphonium compounds, organoaluminum, organotin, organozinc, organotitanium, organozirconium, or organobismuth compounds, and also the compounds known as double-metal-cyanide (DMC) catalysts, as described by way of example in DE 10138216 or in DE 10147712.

It is preferable to use potassium hydroxide, potassium carbonate, potassium hydrogencarbonate, diazabicyclooctane (DABCO), diazabicyclononene (DBN), diazabicycloundecene (DBU), imidazoles, such as imidazole, 1-methylimidazole, or 1,2-dimethylimidazole, titanium tetrabutoxide, titanium tetraisopropoxide, dibutyltin oxide, dibutyltin dilaurate, tin dioctoate, zirconium acetylacetonate, or a mixture thereof.

The amount generally added of the catalyst is from 50 to 10 000 ppm by weight, preferably from 100 to 5000 ppm by weight, based on the amount of the alcohol or alcohol mixture used.

It is moreover also possible to control the intermolecular polycondensation reaction via addition of the suitable catalyst or else via selection of a suitable temperature. The average molecular weight of the hyperbranched polycarbonate can moreover be adjusted by way of the constitution of the starting components and by way of the residence time.

There are various ways of terminating the intermolecular polycondensation reaction. By way of example, the temperature can be lowered to a region in which the reaction stops.

It is moreover possible to deactivate the catalyst, via addition of an acidic component by way of example in the case of basic catalysts, an example being a Lewis acid or an organic or inorganic protic acid.

The high-functionality polycarbonates of the invention are mostly produced in the pressure range from 0.1 mbar to 20 bar, preferably from 1 mbar to 5 bar, in reactors or reactor cascades, which are operated batchwise, semicontinuously, or continuously.

In one further preferred embodiment, the polycarbonates of the invention can comprise further functional groups alongside the functional groups intrinsically present by virtue of the reaction. The functionalization here can take place during the reaction to increase molecular weight or else subsequently, i.e. after the actual polycondensation reaction has ended.

If components which have further functional groups or functional elements, alongside hydroxy or carbonate groups, are added prior to or during the reaction to increase molecular weight, the product comprises a polycarbonate polymer having randomly distributed functionalities which differ from the carbonate, carbamoyl chloride, or hydroxy groups.

Subsequent functionalization can be achieved by an additional process step in which the high-functionality, highly branched or hyperbranched polycarbonate obtained is reacted with a suitable functionalizing reagent which can react with the OH and/or carbonate or carbamoyl chloride groups of the polycarbonate. High-functionality polycarbonates comprising hydroxy groups can moreover also be converted to high-functionality polycarbonate polyether polyols via reaction with alkylene oxides, such as ethylene oxide, propylene oxide, or butylene oxide. Particularly preferred poly-carbonates of the invention here are not only the unfunctionalized polycarbonates but also polycarbonate polyetherols.

The polycarbonate of the invention comprises at least one phosphorus-containing group. This at least one phosphorus-containing group is preferably a group of the general formula (I):

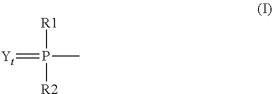

where Y is O or S, t is 0 or 1, R1 and R2, independently of one another, are hydrogen, C₁-C₁₆-alkyl, C₂-C₁₆-alkenyl, C₂-C₁₆-alkynyl, C₁-C₁₆-alkoxy, C₂-C₁₆-alkenoxy, C₂-C₁₆-alkynoxy, C₃-C₁₀-cycloalkyl, C₃-C₁₀-cycloalkoxy, aryl, aryloxy, C₆-C₁₀-aryl-C₁-C₁₆-alkyl, C₆-C₁₀-aryl-C₁-C₁₆-alkoxy, C₁-C₁₆—(S-alkyl), C₂-C₁₆—(S-alkenyl), C₂-C₁₆—(S-alkynyl), C₃-C₁₀—(S-cycloalkyl), S-aryl, NHC₁-C₁₆-alkyl, NHaryl, SR³, COR⁴, COOR⁵, CONR⁶R⁷, and the radicals R³, R⁴, R⁵, R⁶, and R⁷, independently of one another, are C₁-C₁₆-alkyl, C₂-C₁₆-alkenyl, C₂-C₁₆-alkynyl, C₃-C₁₀-cycloalkyl, aryl, aryl-C₁-C₁₆-alkyl, C₁-C₁₆—(S-alkyl), C₂-C₁₆—(S-alkenyl), C₂-C₁₆—(S-alkynyl), or C₃-C₁₀—(S-cycloalkyl), or the radicals R1 and R2 form, together with the phosphorus atom, a ring system.

R1 and R2, identical or different, are preferably C₁-C₁₆-alkyl, C₁-C₁₆-alkoxy, C₃-C₁₀-cycloalkyl, C₃-C₁₀-cycloalkoxy, aryl or aryloxy. Y is preferably O and t is preferably 1.

It is particularly preferable that R1 and R2 are identical, each being phenyl, methoxy-phenyl, tolyl, furyl, cyclohexyl, phenoxy, ethoxy, or methoxy.

To produce the polycarbonates comprising at least one phosphorus-containing group, polycarbonates containing OH groups are reacted, preferably in the presence of a base, with a compound of the general formula (II)

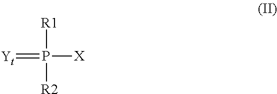

where X is Cl, Br, I, alkoxy, or H, and preferably Cl, and Y, R1 and R2 are defined as above.

The compounds of the formula (II) are known and commercially available, or can be prepared by using synthetic routes well known from the literature. Synthetic routes are described by way of example in Science of Synthesis 42 (2008); Houben Weyl E1-2 (1982); Houben Weyl 12 (1963-1964)].

There are known reactions for producing the polycarbonate of the invention, comprising at least one phosphorus-containing group, by reacting a compound containing OH groups with a compound of the general formula (II). These reactions starting from the phosphorus compound of the formula (II) where X is Cl, Br, or I are described by way of example in WO 2003062251; Dhawan, Balram; Redmore, Derek, J. Org. Chem. (1986), 51(2), 179-183; WO 9617853; Kumar, K. Ananda; Kasthuraiah, M.; Reddy, C. Suresh; Nagaraju, C, Heterocyclic Communications (2003), 9(3), 313-318; Givelet, Cecile; Tinant, Bernard; Van Meervelt, Luc; Buffeteau, Thierry; Marchand-Geneste, Nathalie; Bibal, Brigitte. J. Org. Chem. (2009), 74(2), 652-659.

Reactions where X is alkoxy, an example being transesterification using diphenyl methylphosphonate or triphenyl phosphite, are described by way of example in RU 2101288 and US 2005020800.

Reactions where X is H, an example being the reaction using diphenylphosphine oxide, are described by way of example in Tashev, Emil; Tosheva, Tania; Shenkov, Stoycho; Chauvin, Anne-Sophie; Lachkova, Victoria; Petrova, Rosica; Scopelliti, Rosario; Varbanov, Sabi, Supramolecular Chemistry (2007), 19(7), 447-457.

Examples of suitable bases are metal hydrides, such as sodium hydride, or non-nucleophilic amine bases, such as triethylamine or Hunig's base, bicyclic amines, such as DBU, N-methylimidazole, or N-methylmorpholine, N-methylpiperidine, pyridine, or substituted pyridines, such as lutidine. Triethylamine and N-methylimidazole are particularly preferred. The amounts used of the bases here are generally equimolar. However, the bases can also be used in excess or, if appropriate, as solvent.

The amounts reacted of the starting materials are generally stoichiometric in relation to the desired degree of functionalization. It can be advantageous to use an excess of the phosphorus component with respect to the hydroxy functionalities of the polyol. Random partial phosphorylation can be achieved by using less than the stoichiometric amount of the phosphorus component. The ratios of the starting materials used are preferably such that the phosphorus content of the polycarbonate of the invention, comprising at least one phosphorus-containing group, is at least 3% by weight, with particular preference at least 5% by weight and in particular at least 7% by weight. Another precondition for the stated phosphorus content here, alongside the amount of compound of the formula (II), is the presence of sufficient OH groups in the polycarbonate. These amounts can be adjusted via appropriate conduct of the reaction during production of the polycarbonate, in particular via the proportion of the at least trifunctional polyols, and the reaction time, which controls the conversion and therefore the molecular weight of the resultant polycarbonate. It is possible here that all, or a portion of, the OH groups within the polycarbonate are reacted with the phosphorus component.

The reaction here for producing the polycarbonate of the invention, comprising at least one phosphorus-containing group, is preferably carried out in the presence of a solvent. Suitable solvents for the phosphorylation reactions are inert organic solvents, such as DMSO, halogenated hydrocarbons, such as methylene chloride, chloroform, 1,2-dichloroethane, or chlorobenzene. Solvents which are further suitable are ethers, such as diethyl ether, methyl tert-butyl ether, dibutyl ether, dioxane, or tetrahydrofuran. Solvents which are further suitable are hydrocarbons, such as hexane, benzene, or toluene. Solvents which are further suitable are nitriles, such as acetonitrile or propionitrile. Solvents which are further suitable are ketones, such as acetone, butanone, or tert-butyl methyl ketone. It is also possible to use a mixture of the solvents, and it is also possible to operate without solvent.

The reaction is usually carried out at temperatures of from 0° C. up to the boiling point of the reaction mixture, preferably from 0° C. to 110° C., particularly preferably at from room temperature to 110° C.

The reaction mixtures are worked up in the usual way, e.g. via filtration, mixing with water, separation of the phases and, if appropriate, chromatographic purification of the crude products. The products sometimes take the form of high-viscosity oils, which are freed from volatile constituents, or purified, at reduced pressure and at slightly elevated temperature. To the extent that the resultant products are solids, the purification process can also use recrystallization or digestion.

Another method for phosphorus functionalization consists in the reaction of the polycarbonates of the invention with organophosphorus amides, e.g. neopentylene N,N-dimethylphosphoramidite [cf. Nifant'ev, E. E.; Koroteev, M. P.; Kaziev, G. Z.; Koroteev, A. M.; Vasyanina, L. K.; Zakharova, I. S. Russian Journal of General Chemistry (Translation of Zhurnal Obshchei Khimii) (2003), 73(11), 1686-1690] or BINOL N,N-dimethylphosphoramidite [cf.: Hu, Yuanchun; Liang, Xinmiao; Wang, Junwei; Zheng, Zhuo; Hu, Xinquan, Journal of Organic Chemistry (2003), 68(11), 4542-4545]. The use of P-amidites as phosphorylation reagents in chemistry is well known [cf.: DE 4329533 A1 19950309].

The polycarbonate of the invention, comprising at least one phosphorus-containing group, is used as flame retardant, preferably in plastics. Plastics here comprise all of the known plastics. These comprise thermoplastic molding compositions, for example polyethylene (PE), polypropylene (PP), polyvinyl chloride (PVC), polyamide (PA), polyethylene terephthalate (PET), polyethylene terephthalate glycol (PETG), polybutylene terephthalate (PBT), polyoxymethylene (POM), polycarbonate (PC), polymethyl methacrylate (PMMA), poly(ether) sulfones (PES), thermoplastically processable polyurethane (TPU), polyphenylene oxide (PPO), foamable and/or foamed polypropylene, or a mixture of two or more of said polymers. The polycarbonate of the invention can also be used in crosslinking polymers, for example in polyurethane, e.g. polyurethane foams.

If the polycarbonate of the invention, comprising at least one phosphorus-containing group, is used in thermoplastics, including in thermoplastic polyurethane, the polycarbonate of the invention, comprising at least one phosphorus-containing group, preferably comprises less than 10% of, and with particular preference less than 2% of, and in particular no, free OH groups, based in each case on the entirety of phosphorus-containing groups and OH groups. This is achieved via reaction of the polycarbonate of the invention with the compound of the general formula (I) in an appropriate ratio.

For the purposes of the invention, polyurethane comprises all of the known polyisocyanate polyaddition products. These comprise adducts of isocyanate and alcohol, and they also comprise modified polyurethanes which can comprise isocyanurate structures, allophanate structures, urea structures, carbodiimide structures, uretonimine structures, and biuret structures, and which can comprise further isocyanate adducts. These polyurethanes of the invention comprise in particular solid polyisocyanate polyaddition products, e.g. thermosets, and foams based on polyisocyanate polyaddition products, e.g. flexible foams, semirigid foams, rigid foams, or integral foams, and also polyurethane coatings and binders. For the purposes of the invention, the term polyurethanes also includes polymer blends comprising polyurethanes and further polymers, and also foams made of said polymer blends. It is preferable that the polycarbonates of the invention, comprising at least one phosphorus-containing group, are used in producing polyurethane foams.

For the purposes of the invention, polyurethane foams are foams to DIN 7726. The compressive stress value for flexible polyurethane foams of the invention at 10% compression, or the compressive strength of these foams to DIN 53 421/DIN EN ISO 604, is 15 kPa or less, preferably from 1 to 14 kPa, and in particular from 4 to 14 kPa. The compressive stress value for semirigid polyurethane foams of the invention at 10% compression to DIN 53 421/DIN EN ISO 604 is from greater than 15 to less than 80 kPa. The open-cell factor to DIN ISO 4590 of semirigid polyurethane foams of the invention and of flexible polyurethane foams of the invention is preferably greater than 85%, particularly preferably greater than 90%. Further details concerning flexible polyurethane foams of the invention and semirigid polyurethane foams of the invention are found in “Kunststoffhandbuch, Band 7, Polyurethane” [Plastics handbook, volume 7, Polyurethanes], Carl Hanser Verlag, 3rd edition, 1993, chapter 5.

The compressive stress value for rigid polyurethane foams of the invention at 10% compression is greater than or equal to 80 kPa, preferably greater than or equal to 120 kPa, particularly preferably greater than or equal to 150 kPa. The closed-cell factor to DIN ISO 4590 for the rigid polyurethane foam is moreover greater than 80%, preferably greater than 90%. Further details concerning rigid polyurethane foams of the invention are found in “Kunststoffhandbuch, Band 7, Polyurethane” [Plastics handbook, volume 7, Polyurethanes], Carl Hanser Verlag, 3rd edition, 1993, chapter 6.

For the purposes of this invention, elastomeric polyurethane foams are polyurethane foams to DIN 7726, where these exhibit no residual deformation beyond 2% of their initial thickness 10 minutes after brief deformation amounting to 50% of their thickness to DIN 53 577. This foam can be a rigid polyurethane foam, a semirigid polyurethane foam, or a flexible polyurethane foam.

Integral polyurethane foams are polyurethane foams to DIN 7726 having a marginal zone in which the density is higher than in the core, as a result of the shaping process. The overall density here averaged over the core and the marginal zone is preferably above 100 g/L. For the purposes of the invention, integral polyurethane foams can again be rigid polyurethane foams, semirigid polyurethane foams, or flexible polyurethane foams. Further details concerning the integral polyurethane foams of the invention are found in “Kunststoffhandbuch, Band 7, Polyurethane” [Plastics handbook, volume 7, Polyurethanes], Carl Hanser Verlag, 3rd edition, 1993, chapter 7.

Polyurethanes are obtained here by mixing isocyanates (a) with polyols (b), with a polycarbonate according to any of claims 1 to 8 (c) and, if appropriate, with blowing agent (d), with catalyst (e), and with other auxiliaries and additives (f) to give a reaction mixture and permitting completion of the reaction.

Polyisocyanate components (a) used for producing the polyurethanes of the invention comprise all of the polyisocyanates known for producing polyurethanes. These comprise the aliphatic, cycloaliphatic, and aromatic di- or polyfunctional isocyanates known from the prior art, and also any desired mixtures thereof. Examples are diphenylmethane 2,2″-, 2,4″-, and 4,4″-diisocyanate, the mixtures of monomeric diphenylmethane diisocyanates and of diphenylmethane diisocyanate homologues having a larger number of rings (polymer MDI), isophorone diisocyanate (IPDI) and its oligomers, tolylene 2,4- or 2,6-diisocyanate (TDI) and mixtures of these, tetramethylene diisocyanate and its oligomers, hexamethylene diisocyanate (HDI) and its oligomers, naphthylene diisocyanate (NDI), and mixtures thereof.

It is preferable to use tolylene 2,4- and/or 2,6-diisocyanate (TDI) or a mixture of these, monomeric diphenylmethane diisocyanates and/or diphenylmethane diisocyanate homologues having a larger number of rings (polymer MDI) and mixtures of these. Other possible isocyanates are given by way of example in “Kunststoffhandbuch, Band 7, Polyurethane” [Plastics handbook, volume 7, Polyurethanes], Carl Hanser Verlag, 3rd edition, 1993, chapters 3.2 and 3.3.2.

Polyisocyanate component (a) can be used in the form of polyisocyanate prepolymers. Said polyisocyanate prepolymers are obtainable by reacting an excess of polyisocyanates (constituent (a-1)) described above with polyols (constituent (a-2)), for example at temperatures of from 30 to 100° C., preferably about 80° C., to give the prepolymer.

Polyols (a-2) are known to the person skilled in the art and are described by way of example in “Kunststoffhandbuch, 7, Polyurethane” [Plastics handbook, volume 7, Polyurethanes], Carl Hanser Verlag, 3rd edition, 1993, chapter 3.1. By way of example, therefore, the polyols used can also comprise the polyols described below under (b). In one particular embodiment here, the polyisocyanate prepolymer can also comprise the polycarbonate of the invention, comprising at least one phosphorus-containing group.

Polyols that can be used comprise all of the compounds (b) known for polyurethane production and having at least two reactive hydrogen atoms, examples being those having functionality of from 2 to 8 and molecular weight of from 400 to 15 000. It is therefore possible by way of example to use polyols selected from the group of the polyether polyols, polyester polyols, and mixtures thereof.

By way of example, polyetherols are produced from epoxides, such as propylene oxide and/or ethylene oxide, or from tetrahydrofuran, by using starter compounds containing active hydrogen, e.g. aliphatic alcohols, phenols, amines, carboxylic acids, water, or compounds based on natural materials, e.g. sucrose, sorbitol, or mannitol, with use of a catalyst. Mention may be made here of basic catalysts or double-metal-cyanide catalysts, as described by way of example in PCT/EP2005/010124, EP 90444 or WO 05/090440.

By way of example, polyesterols are produced from aliphatic or aromatic dicarboxylic acids and from polyfunctional alcohols, from polythioether polyols, from polyesteramides, from polyacetals containing hydroxy groups, and/or from aliphatic polycarbonates containing hydroxy groups, preferably in the presence of an esterification catalyst. Other possible polyols are given by way of example in “Kunststoffhandbuch, Band 7, Polyurethane” [Plastics handbook, volume 7, Polyurethanes], Carl Hanser Verlag, 3rd edition, 1993, chapter 3.1.

Polyols (b) also comprise chain extenders and crosslinking agents. The molar mass of chain extenders and crosslinking agents is less than 400 g/mol, and the term used here for molecules having two hydrogen atoms reactive toward isocyanate is chain extenders, while the term used for molecules having more than two hydrogens reactive toward isocyanate is crosslinking agents. Although it is possible here to omit the chain extender or crosslinking agent, addition of chain extenders or crosslinking agents or else, if appropriate, a mixture thereof has proven advantageous for modifying mechanical properties, e.g. hardness.

If chain extenders and/or crosslinking agents are used, it is possible to use the chain extenders and/or crosslinking agents that are known for the production of polyurethanes. These are preferably low-molecular-weight compounds having functional groups reactive toward isocyanates, examples being glycerol, trimethylol-propane, glycol, and diamines. Other possible low-molecular-weight chain extenders and/or crosslinking agents are given by way of example in “Kunststoffhandbuch, Band 7, Polyurethane” [Plastics handbook, volume 7, Polyurethanes], Carl Hanser Verlag, 3rd edition, 1993, chapters 3.2 and 3.3.2.

A polycarbonate of the invention, comprising at least one phosphorus-containing group, is moreover used as component (c). The proportion of polycarbonate comprising at least one phosphorus-containing group (c), hereinafter also termed polycarbonate (c), is subject to no restriction here and depends primarily on the degree of flame retardancy to be achieved. The proportion of polycarbonate here can by way of example vary from 0.1 to 50% by weight, preferably from 1 to 40% by weight, and particularly preferably from 2 to 30% by weight, based in each case on the total weight of components (a) to (e). The phosphorus content in the finished polyurethane here is preferably from 0.01 to 10% by weight, particularly preferably from 0.05 to 5% by weight, and in particular from 0.1 to 5% by weight, based in each case on the total weight of the polyurethane.

The reaction mixtures of the invention preferably also comprise blowing agents (d) if the polyurethane is intended to take the form of polyurethane foam. It is possible here to use any of the blowing agents known for producing polyurethanes. These can comprise chemical and/or physical blowing agents. These blowing agents are described by way of example in “Kunststoffhandbuch, Band 7, Polyurethane” [Plastics handbook, volume 7, Polyurethanes], Carl Hanser Verlag, 3rd edition, 1993, chapter 3.4.5. The term chemical blowing agents is used here for compounds which form gaseous products via reaction with isocyanate. Examples of these blowing agents are water and carboxylic acids. The term physical blowing agents is used here for compounds which have been dissolved or emulsified in the starting materials for polyurethane production and which evaporate under the conditions of polyurethane formation. By way of example, these are hydrocarbons, halogenated hydrocarbons, and other compounds, e.g. perfluorinated alkanes, such as perfluorohexane, fluorochlorocarbons, and ethers, esters, ketones, acetals, and/or a liquid form of carbon dioxide. The amount used of the blowing agent here can be as desired. The amount used of the blowing agent is preferably such that the density of the resultant polyurethane foam is from 10 to 1000 g/L, particularly preferably from 20 to 800 g/L, and in particular from 25 to 200 g/L.

Catalysts (e) used can comprise any of the catalysts usually used for polyurethane production. These catalysts are described by way of example in “Kunststoffhandbuch, Band 7, Polyurethane” [Plastics handbook, volume 7, Polyurethanes], Carl Hanser Verlag, 3rd edition, 1993, chapter 3.4.1. Examples of those used here are organometallic compounds, preferably organotin compounds, e.g. stannous salts of organic carboxylic acids, for example stannous acetate, stannous octoate, stannous ethylhexanoate, and stannous laurate, and the dialkyltin(IV) salts of organic carboxylic acids, e.g. dibutyltin diacetate, dibutyltin dilaurate, dibutyltin maleate, and dioctyltin diacetate, and also bismuth carboxylates, such as bismuth(III) neodecanoate, bismuth 2-ethylhexanoate, and bismuth octanoate, or a mixture. Other possible catalysts are basic amine catalysts. Examples of these are amidines, such as 2,3-dimethyl-3,4,5,6-tetrahydropyrimidine, tertiary amines, such as triethylamine, tributylamine, dimethylbenzylamine, N-methyl- and N-ethyl-N-cyclohexylmorpholine, N,N,N′,N′-tetramethylethylenediamine, N,N,N′,N′-tetramethylbutanediamine, N,N,N′,N′-tetramethylhexanediamine, pentamethyldiethylenetriamine, tetramethyldiaminoethyl ether, bis(dimethylaminopropyl)urea, dimethylpiperazine, 1,2-dimethylimidazole, 1-aza-bicyclo[3.3.0]octane, and preferably 1,4-diazabicyclo[2.2.2]octane, and alkanolamine compounds, such as triethanolamine, triisopropanolamine, N-methyl- and N-ethyl-diethanolamine, and dimethylethanolamine. The catalysts can be used individually or in the form of mixtures. If appropriate, the catalysts (e) used comprise mixtures of metal catalysts and of basic amine catalysts.

Particularly if a relatively large excess of polyisocyanate is used, other catalysts that can be used are: tris(dialkylaminoalkyl)-s-hexahydrotriazines, preferably tris(N,N-dimethylaminopropyl)-s-hexahydrotriazine, tetraalkylammonium hydroxides, such as tetramethylammonium hydroxide, alkali metal hydroxides, such as sodium hydroxide, and alkali metal alcoholates, such as sodium methoxide and potassium isopropoxide, and also the alkali metal or ammonium salts of carboxylic acids, e.g. potassium formate or ammonium formate, or the corresponding acetates or octoates.

Examples of the concentration of the catalysts (e) that can be used are from 0.001 to 5% by weight, in particular from 0.05 to 2% by weight in the form of catalyst or catalyst combination, based on the weight of component (b).

It is also possible to use auxiliaries and/or additives (f). It is possible here to use any of the auxiliaries and additives known for producing polyurethanes. By way of example, mention may be made of surface-active substances, foam stabilizers, cell regulators, release agents, fillers, dyes, pigments, flame retardants, hydrolysis stabilizers, and fungistatic and bacteriostatic substances. These substances are described by way of example in “Kunststoffhandbuch, Band 7, Polyurethane” [Plastics handbook, volume 7, Polyurethanes], Carl Hanser Verlag, 3rd edition, 1993, chapter 3.4.4 and 3.4.6 to 3.4.11.

When producing the polyurethane of the invention, the amounts reacted of the polyisocyanates (a), the polyols (b), the polycarbonates (c) and, if appropriate, the blowing agents (d) are generally such that the equivalence ratio of NCO groups of the polyisocyanates (a) to the total number of reactive hydrogen atoms in components (b), (c), and, if appropriate, (d) is from 0.75 to 1.5:1, preferably from 0.80 to 1.25:1. If the cellular plastics comprise at least some isocyanurate groups, the ratio used of NCO groups of the polyisocyanates (a) to the total number of reactive hydrogen atoms in component (b), (c) and, if appropriate, (d) and (f) is usually from 1.5 to 20:1, preferably from 1.5 to 8:1. A ratio of 1:1 here corresponds to an isocyanate index of 100.

There is respectively very little quantitative and qualitative difference between the specific starting materials (a) to (f) used for producing polyurethanes of the invention when the polyurethane to be produced of the invention is a thermoplastic polyurethane, a flexible foam, a semirigid foam, a rigid foam, or an integral foam. By way of example, therefore, the production of solid polyurethanes uses no blowing agents, and for thermoplastic polyurethane the starting materials used are predominantly strictly difunctional. It is also possible by way of example to use the functionality and the chain length of the relatively high-molecular-weight compound having at least two reactive hydrogen atoms to vary the elasticity and hardness of the polyurethane of the invention. This type of modification is known to the person skilled in the art.

By way of example, the starting materials for producing a solid polyurethane are described in EP 0989146 or EP 1460094, the starting materials for producing a flexible foam are described in PCT/EP2005/010124 and EP 1529792, the starting materials for producing a semirigid foam are described in “Kunststoffhandbuch, Band 7, Polyurethane” [Plastics handbook, volume 7, Polyurethanes], Carl Hanser Verlag, 3rd edition, 1993, chapter 5.4, the starting materials for producing a rigid foam are described in PCT/EP2005/010955, and the starting materials for producing an integral foam are described in EP 364854, U.S. Pat. No. 5,506,275, or EP 897402. In each case, the polycarbonate (c) is then also added to the starting materials described in said documents.

In one embodiment of the invention here, a polycarbonate (c) is used which has less than 10% of, particularly preferably less than 2% of, and in particular no, free OH groups, based in each case on the entirety of phosphorus-containing groups and OH groups.

In another embodiment of the present invention, the polycarbonate (c) has OH groups. Here, the polycarbonate (c) is preferably adapted in relation to functionality and OH number in such a way that there is only slight impairment of the mechanical properties of the resultant polymer, or preferably indeed an improvement therein. At the same time, change to the processing profile is minimized. This type of adaptation can by way of example be achieved in that the OH number and functionality of the compound (c) are within the region of the OH number and functionality of the polyol used for polyurethane production.

If the polycarbonate (c) has OH groups, the production of flexible polyurethane foams preferably uses, as polycarbonate (c), a compound which has an OH number of from 2 to 100 mg KOH/g, particularly preferably from 10 to 80 mg KOH/g, and in particular from 20 to 50 mg KOH/g, with an OH functionality which is preferably from 2 to 4, particularly preferably from 2.1 to 3.8, and in particular from 2.5 to 3.5.

If the polycarbonate (c) has OH groups, the production of rigid polyurethane foams preferably uses, as polycarbonate (c), a compound which has an OH number which is preferably from 2 to 800 mg KOH/g, particularly preferably from 50 to 600 mg KOH/g, and in particular from 100 to 400 mg KOH/g, with an OH functionality which is preferably from 2 to 8, particularly preferably from 2 to 6.

If the polycarbonate (c) has OH groups, the production of thermoplastic polyurethane (TPU) preferably uses, as polycarbonate (c), a compound which has an OH number of from 2 to 800 mg KOH/g, particularly preferably from 10 to 600 mg KOH/g, and in particular from 20 to 400 mg KOH/g, with an OH functionality which is preferably from 1.8 to 2.2, particularly preferably from 2.9 to 2.1, and in particular 2.0.

If a polyisocyanurate foam is produced, using a ratio of NCO groups of the polyisocyanates (a) to the total number of reactive hydrogen atoms in component (b), (c), and, if appropriate, (d) and (f) which is from 1.5 to 20:1, the OH functionality of component (c) is preferably from 2 to 3, with an OH number which is preferably from 20 to 800 mg KOH/g, particularly preferably from 50 to 600 mg KOH/g, and in particular from 100 to 400 mg KOH/g.

However, it is also possible in all cases to use any of the polycarbonates (c).

It is preferable here that the polycarbonate comprising at least one phosphorus-containing group (c) is soluble in the polyols (b). “Soluble” here means that after 24 h of standing at 50° C. no second phase that is visible to the naked eye has formed in a mixture of polyol component (b) and component (c) in the ratio corresponding to the amount subsequently used for producing the polyurethane. Solubility here can by way of example be improved via functionalization of component (c) or, respectively, the polycarbonate of the invention, for example by using alkylene oxide.

Examples will be used below to illustrate the invention.

### Synthesis of a Polycarbonate

2400 g of trimethylolpropane×1.2 propylene oxide, 1417.5 g of diethyl carbonate, and 0.6 g of K₂CO₃ as catalyst (250 ppm of catalyst, based on the mass of alcohol) were used as initial charge in a 4 L three-necked flask equipped with stirrer, reflux condenser, and internal thermometer. The mixture was heated to from 120° C. to 140° C. and stirred at this temperature for 2 h. As the reaction time increased, the temperature of the reaction mixture decreased because of the onset of evaporative cooling by the ethanol liberated. The reflux condenser was then replaced by an inclined condenser, the ethanol was removed by distillation, and the temperature of the reaction mixture was slowly increased up to 160° C. 795 g of ethanol were obtained here.

### Analysis:

The reaction products were then analyzed by gel permeation chromatography, with dimethylacetamide as eluent, and polymethyl methacrylate (PMMA) as standard. The values determined were:

Mₙ: 827 g/mol
M_{w}: 1253 g/mol
The OH number was determined to DIN 53240:
OH number: 416 mg KOH/g
Phosphorylation of the Polycarbonate with Diphenylphosphinyl Chloride:

403.5 g of the highly branched polycarbonate from example 1 were dissolved in 400 mL of toluene with argon inertization, in a 2 L four-necked flask equipped with Teflon stirrer, reflux condenser, thermometer, and dropping funnel. 379.5 g of triethylamine were added all at once. The mixture was heated to 90° C., and 710.5 g of diphenylphosphinyl chloride were added dropwise within a period of 120 minutes. Stirring of the mixture was then continued for 12 hours at 80° C. Conversion was controlled by means of quantitative conversion of the acid chloride, as indicated by ³¹P NMR.

After cooling to room temperature, the reaction mixture was extracted twice with 1 liter of 10% strength by weight sodium bicarbonate solution, and once with 500 mL of the water. The organic phase was dried over sodium sulfate. The product (polymer 1) was isolated in the form of dark yellow oil after removal of the volatile constituents in vacuo.

### Analysis:

OH number: 2 mg KOH/g

Polyurethane foams were produced as in table 1 and table 2 by first mixing all of the components except for metal catalysts and isocyanate. Metal catalysts were then added if appropriate and likewise incorporated by stirring. The isocyanate was weighed out separately and then added to the polyol component. The mixture was mixed until the reaction began, and was then poured into a metal box lined with plastic film. The total size of the batch was in each case 1800 g. The foam completed its reaction overnight and was separated by sawing to give test specimens.

TABLE 1

| | | Reference 1 | Reference 2 | Reference 3 | Reference 4 | Reference 5 |
|---|---|---|---|---|---|---|
| Polyol 1 | | 66.70 | 66.70 | 66.70 | 66.70 | 66.7 |
| Polyol 2 | | 33.30 | 33.30 | 33.30 | 33.30 | 33.3 |
| Tegostab B8681 | | 0.50 | 0.50 | 0.50 | 0.50 | 0.5 |
| Catalyst system 1 | | 0.42 | 0.38 | 0.45 | 0.35 | 0.45 |
| Diethanolamine (80%) | | 1.49 | 1.49 | 1.49 | 1.49 | 1.49 |
| Ortegol 204 | | 1.50 | 1.50 | 1.50 | 1.50 | 1.50 |
| Catalyst system 2 | | | | | | |
| Glycerol | | | | | | |
| Water | | 1.90 | 2.10 | 2.10 | 2.10 | 2.10 |
| Reofos ® TPP | | | 8.00 | | | |
| Fyroflex ® BDP | | | | 8.00 | | |
| Fyrol ® 6 | | | | | 8.00 | |
| TCPP | | | | | | 8.00 |
| Isocyanate 1 | | 100 | 100 | 100 | 100 | 100 |
| P content of foam | [%] | 0 | 0.5 | 0.5 | 0.6 | 0.5 |
| CI content of foam | [%] | 0 | 0 | 0 | 0 | 1.7 |
| Density | [kg/m³] | 37.2 | 36 | 32.8 | 35.5 | 35.4 |
| Compressive strength at 40% | [kPa] | 3.5 | 4.2 | 3.4 | 4.8 | 3.7 |
| Rebound resilience | [%] | 53 | 54 | 54 | 49 | 55 |
| Permeability to air | [dm³/s] | 0.567 | 0.598 | 1.153 | 0.695 | 0.667 |
| California TB 117 A | | | | | | |
| Average carbonized distance | [cm] | 262 | 134 | 207 | 155 | 112 |
| Maximum carbonized distance | [cm] | 306 | 147 | 256 | 176 | 128 |
| Average afterflame time | [s] | 29 | 0 | 25 | 1 | 0 |
| Maximum afterflame time | [s] | 42 | 0 | 68 | 2 | 0 |
| Average afterglow time | [s] | 0 | 0 | 0 | 0 | 0 |
| Result | | failed | passed | failed | failed | passed |

TABLE 2

| | | Example 1 | Example 2 | Example 3 | Example 4 | Example 5 |
|---|---|---|---|---|---|---|
| Polyol 1 | | 66.70 | 66.70 | 66.70 | 66.70 | 66.70 |
| Polyol 2 | | 33.30 | 33.30 | 33.30 | 33.30 | 33.30 |
| Tegostab B8681 | | 0.50 | 0.50 | 0.50 | 0.50 | 0.50 |
| Catalyst system 1 | | 0.4 | 0.45 | | | |
| Diethanolamine (80%) | | 1.49 | 1.49 | 1.49 | 1.49 | 1.49 |
| Ortegol 204 | | 1.50 | 1.50 | 1.50 | 1.50 | |
| Catalyst system 2 | | | | 1.00 | 1.00 | 0.65 |
| Glycerol | | | | | | |
| Water | | 2.20 | 2.00 | 2.45 | 2.70 | 2.80 |
| HB-polyol 1 | | 12.00 | | | | |
| HB-polyol 2 | | | 12.00 | | | |
| HB-polyol 3 | | | | 12.00 | | |
| HB-polyol 4 | | | | | 12.00 | |
| HB-polyol 5 | | | | | | 12.00 |
| Isocyanate 1 | | 100 | 100 | 100 | 100 | 100 |
| P content of foam | [%] | 0.5 | 0.4 | 0.7 | 0.7 | 0.6 |
| CI content of foam | [%] | 0 | 0 | 0 | 0 | 0 |
| mechanical properties | | | | | | |
| Density | [kg/m³] | 32.3 | 36.7 | 37.4 | 32.3 | 37.5 |
| Compressive strength at 40% | [kPa] | 3 | 3.5 | 5.8 | 4.2 | 3.1 |
| Rebound resilience | [%] | 53 | 52 | 50 | 51 | 52 |
| Permeability to air | [dm³/s] | 1.133 | 0.816 | 0.518 | 0.788 | 0.429 |
| California TB 117 A | | | | | | |
| Average carbonized distance | [cm] | 138 | 129 | 117 | 120 | 132 |
| Maximum carbonized distance | [cm] | 152 | 145 | 127 | 124 | 150 |
| Average afterflame time | [s] | 0 | 1 | 0 | 0 | 1 |
| Maximum afterflame time | [s] | 0 | 3 | 0 | 0 | 0 |
| Average afterglow time | [s] | 0 | 0 | 0 | 0 | 0 |
| Result | | passed | passed | passed | passed | passed |

Key:
Polyol 1: polyoxypropylene polyoxyethylene polyol; OH number: 35; functionality: 2.7
Polyol 2: Graft polyol based on styrene-acrylonitrile; solids content: 45%; polyoxypropyleneoxyethylene polyol; OH number: 20; functionality: 2.7
Catalyst system 1: standard catalyst system made of metal catalyst and amine catalyst
Catalyst system 2: amine catalysts partially capped by formic acid
Isocyanate 1: mixture of toluene 2,4- and 2,6-diisocyanate
HB polyol 1: hyperbranched polycarbonate, partially reacted with chloro-diphenyl phosphate; OH number: 12; 6.6% by weight of P
HB polyol 2: hyperbranched polycarbonate, reacted with chlorodiphenyl phosphate; OH number: 0; 4.8% by weight of P
HB polyol 3: hyperbranched polycarbonate, partially reacted with chloro-diphenylphosphinyl chloride; OH number: 2; 9.1% by weight of P
HB polyol 4: hyperbranched polycarbonate, partially reacted with chloro-diphenylphosphinyl chloride; OH number: 19; 9.0% by weight of P
HB polyol 5: hyperbranched polycarbonate, partially reacted with chloro-diphenylphosphine oxide; OH number: 43; 7.7% by weight of P
Reofos ® TPP: triphenyl phosphate; 9.5% by weight of P (Chemtura)
Fyrolflex ® BDP: bisphenol A bis(diphenyl phosphate)/triphenyl phosphate); from 8.9 to 9% by weight of P (Supresta)
Fyrol ® 6: diethyl bis(2-hydroxyethylamino)methanephosphonate; 12% by weight of P (Supresta)

The following methods were used to determine properties:
Density in kg/m³: DIN EN ISO 845
Compressive strength in kPa: DIN EN ISO 3386
Rebound resilience in %: DIN EN ISO 8307
Permeability to air in dm³/s: DIN EN ISO 7231
Flame retardancy: California TB 117 A

From the tables it can be seen that the halogen-free flexible polyurethane foams of the invention exhibit very good flame retardancy, similar to or better than that of the comparative foams which used commercially available samples with similar or even higher phosphorus content. It is also found that the mechanical properties of the foams are improved rather than impaired, despite the presence of the incorporatable flame retardants.

Comparative example 4 shows that this is not necessarily the case with commercial samples, and here elasticity is markedly impaired. At low densities, the novel structures have better effect than the commercial samples of comparative example 3, while phosphorus content is the same or insignificantly higher. Although a polyurethane foam using triphenyl phosphate as flame retardant (comparative example 2) exhibits the same qualities in respect of flame retardancy and mechanical properties as the hyperbranched, phosphorus-containing polycarbonates, the low-molecular-weight compound here contributes significantly to emissions from the foam. If the results of table 2 are compared with the result achieved using the commercial flame retardant that is most widely used (tris(chloroisopropyl)phosphate (TCPP)), the results are seen to be fully comparable. The foam using trichloroisopropyl phosphate here has the same phosphorus content, but also comprises 1.7% of chlorine. This is therefore not a halogen-free method of achieving flame retardancy. The results show that phosphorylated hyperbranched polycarbonates are suitable flame retardants for replacing the halogenated material tris(chloroisopropyl)phosphate. Surprisingly, despite the lack of chlorine, there is no need here to increase phosphorus content in order to achieve the same effects.

A rigid polyurethane foam was also produced as in table 3:

TABLE 3

| | Example 5 | Reference 6 |
|---|---|---|
| Polyol 3 | 65 | 65 |
| Polyol 4 | 10 | 10 |
| Stabilizer 1 | 2 | 2 |
| HB polyol 5 | 25 | |
| Trichloroisopropyl phosphate | | 25 |
| Blowing agent 1 | 9 | 9 |
| Blowing agent 2 | 1.6 | 1.6 |
| Catalyst 3 | 1.2 | 1.2 |
| Catalyst 4 | 2 | 2 |
| Isocyanate 2 | 190 | 190 |
| Density (g/L) | 45 | 45 |
| Fiber time (s) | 45 | 45 |
| Tack-free time (s) | 65 | 64 |
| BKZ 5 test | passed | passed |

The starting materials used were as follows:
Polyol 3: esterification product of phthalic anhydride and diethylene glycol, OHN=220 mg KOH/g
Polyol 4: polyethylene glycol, OHN=200 mg KOH/g

### Stabilizer 1: Tegostab® B 8467 (Evonik Goldschmidt GmbH)

HB polyol 5: Hyperbranched polycarbonate, partially reacted with chlorodiphenyl phosphate; OHN=19 mg KOH/g, phosphorus content 10.3% by weight
Blowing agent 1: n-pentane
Blowing agent 2: formic acid (85% by weight)
Blowing agent 3: mixture of water and dipropylene glycol, ratio by weight 3:2
Catalyst 1: potassium formate (36% by weight in ethylene glycol)
Catalyst 2: bis(2-dimethylaminoethyl)ether (70% by weight in dipropylene glycol)
Isocyanate 1: polymeric MDI

The tack-free time is defined here as the period between the start of the mixing process and the juncture at which there is almost no tack discernible when a rod or the like touches the surface of the foam. The tack-free time is a measure of the effectiveness of the urethane reaction.

BKZ5 test: Flame test for determining flammability of construction materials to the Swiss testing and classification standard issued by the Vereinigung Kantonaler Feuerversicherungen [Association of Cantonal Fire Insurers].

The rigid PU foams were produced by mixing the polyols used, stabilizers, flame retardants, catalysts, and blowing agents, and then mixing these with the isocyanate and foaming to give the rigid PU foam.

From table 3 it can be seen that when phosphorus-containing polycarbonates of the invention are used it is possible to pass the BKZ5 test without any effect on the reactivity of the foam system. However, unlike conventional commercial flame retardants such as TCPP, the flame retardants of the invention are halogen-free.

1. A polycarbonate comprising at least one phosphorus-containing group.

2. The polycarbonate according to claim 1, where the phosphorus-containing group is a unit of the general formula 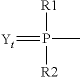
and
Y is O or S,
t is 0 or 1,
R1 and R2, independently of one another, are hydrogen, C₁-C₁₆-alkyl, C₂-C₁₆-alkenyl, C₂-C₁₆-alkynyl, C₁-C₁₆-alkoxy, C₂-C₁₆-alkenoxy, C₂-C₁₆-alkynoxy, C₃-C₁₀-cycloalkyl, C₃-C₁₀-cycloalkoxy, aryl, aryloxy, C₆-C₁₀-aryl-C₁-C₁₆-alkyl, C₆-C₁₀-aryl-C₁-C₁₆-alkoxy, C₁-C₁₆—(S-alkyl), C₂-C₁₆—(S-alkenyl), C₂-C₁₆—(S-alkynyl), C₃-C₁₀—(S-cycloalkyl), S-aryl, NHC₁-C₁₆-alkyl, NHaryl, SR³, COR⁴, COOR⁵, CONR⁶R⁷, and the radicals R³, R⁴, R⁶, R⁶, and R⁷, independently of one another, are C₁-C₁₆-alkyl, C₂-C₁₆-alkenyl, C₂-C₁₆-alkynyl, C₃-C₁₀-cycloalkyl, aryl, aryl-C₁-C₁₆-alkyl, C₁-C₁₆—(S-alkyl), C₂-C₁₆—(S-alkenyl), C₂-C₁₆—(S-alkynyl), or C₃-C₁₀—(S-cycloalkyl), or the radicals R1 and R2 form, together with the phosphorus atom, a ring system.

3. The polycarbonate according to claim 2, where R1 is the same as R2, and each of R1 and R2 is methoxyphenyl, tolyl, furyl, cyclohexyl, phenyl, phenoxy, ethoxy, or methoxy.

4. The polycarbonate according to any of claims 1 to 3, which comprises at least 3% by weight of phosphorus.

5. The polycarbonate according to any of claims 1 to 4, which comprises no OH groups.

6. The polycarbonate according to any of claims 1 to 4, comprising at least one OH group.

7. The polycarbonate according to claim 6, which has an OH number of from 2 to 800 mg KOH/g.

8. The polycarbonate according to any of claims 1 to 7, comprising propylene oxide units and/or ethylene oxide units.

9. The polycarbonate according to any of claims 1 to 8, which is a hyperbranched polycarbonate.

10. The polycarbonate according to any of claims 1 to 9, which comprises no aromatic constituents in the carbonate skeleton.

11. The use of a polycarbonate according to any of claims 1 to 10 as flame retardant.

12. A plastic comprising a polycarbonate according to any of claims 1 to 10.

13. A process for producing a polyurethane, by mixing isocyanates (a) with polyols (b), with a polycarbonate according to any of claims 1 to 10 (c) and, if appropriate, with blowing agent (d), with catalyst (e), and with other auxiliaries and additives (f) to give a reaction mixture and permitting completion of the reaction to give the polyurethane.

14. The process for producing a polyurethane, according to claim 13, where the poly-urethane is a polyurethane foam.

15. A polyurethane obtainable according to claim 13 or 14.
